(19) United States
(12) Patent Application Publication
(10) Pub. No.: US 2021/0298681 A1
(43) Pub. Date: Sep. 30, 2021

(54) SYSTEM AND METHOD FOR WALKER GAIT TRACKER

(71) Applicants: George Kolton, St. Petersburg, FL (US); William D Furlong, II, Largo, FL (US)

(72) Inventors: George Kolton, St. Petersburg, FL (US); William D Furlong, II, Largo, FL (US)

(21) Appl. No.: 16/833,566

(22) Filed: Mar. 28, 2020

(51) Int. Cl. A61B 5/00; A61B 5/0205; A61B 5/024; A61B 5/145

(52) U.S. Cl. CPC A61B 5/6887; A61B 5/14542; A61B 5/02438; A61B 5/0205

(57) ABSTRACT

A system and method for measuring a patient's gait while using a tracker coupled to a walker is disclosed. The system has a console display unit having a clamping mechanism for attaching to the walker, a measuring wheel assembly, and one or more distance measurements based upon a number of electronic pluses received from the measuring wheel assembly and the known circumference of the measuring wheel. Only one of the plurality of counters and corresponding distance display are active at any one time. The console display includes a plurality of distance displays, a plurality of counters each coupled to one of the plurality of distance displays, a timer display, a timer circuit having a start/stop input. and control logic. The measuring wheel assembly has a measuring wheel with a known circumference and attachment clamp coupled to the assembly. The measuring wheel assembly is coupled to a leg of the walker at a height that allows the measuring wheel to rest on the floor and rotate freely. The measuring wheel assembly generates an electronic pulse corresponding to a rotation of plurality of equal length segments on the circumference of the measuring wheel.

### TECHNICAL FIELD

This application relates in general to a system and method for measuring a patient's gait while using a walker.

### BACKGROUND

People are often placed into various rehabilitation situations in order to address functional mobility deficits and increased risk from falling while standing/walking. When starting physical therapy, evaluating therapists are required to establish and accurately document starting walking (gait) abilities, in terms of objective measures including walking distances and time needed to traverse a specific distance, very often with a walker of some type. Therapists are also required to accurately measure and document the objective details of every subsequent walk performed during daily treatment sessions and during the final treatment session at time of discharge from therapy.

These objective measures are critical for documentation of progress being made, or not made, toward specific, objective walking goals established after baseline measures are taken during the patient's initial therapy evaluation session. These measures are also critical for progress-based justification of therapy services, as assessed by rehabilitation service payers such as Medicare and other insurers. When performing gait evaluation and training, therapists will typically use a distance measuring wheel while repeatedly retracing each walk a patient just performed. Many therapists skip this tedious process by estimating the distances patients walk. Retracing every single walk a patient just performed while measuring the distance with a distance measuring wheel, is inefficient, inconvenient and time consuming. While skipping the use of a measuring wheel and estimating distances can lead to drastic inaccuracies. Governing bodies and therapy service payers do not look favorably upon such “estimating”. The present invention attempts to address the existing limitations in for measuring a patient's gait while using a walker according to the principles and example embodiments disclosed herein.

### SUMMARY

In accordance with the present invention, the above and other problems are solved by providing a system and method for measuring a patient's gait while using a walker, and more specifically for providing a digital walker distance counter for measuring a patient's gait while using a walker.

In one embodiment, the present invention is a system for measuring a patient's gait while using a walker. The system includes a measuring wheel, a connection cable, and a control and display console. The measuring wheel generates a pre-defined number of equally spaced pulses for each rotation of the measuring wheel. The control and display console includes a plurality of counters that keep track of pulses received from the measuring wheel while the tracker is enabled. The distance traveled may be easily calculated and displayed from a measurement of the circumference of the measuring wheel and the number of pulses received.

In another embodiment, the present invention is a method for measuring a patient's gait while using a walker. The method receives a pulse from the measuring wheel, increments a counter while the tracker is enabled, calculates a distance traveled from a measurement of the circumference of the measuring wheel and the number of pulses received, and displays the measured distance to a user on the console.

The great utility of the invention is that a system and method for measuring a patient's gait while using a walker assists healthcare providers and patients to measure and monitor a patient's gait while rehabbing. The present invention eliminates inefficiencies and inaccuracies in the way therapists measure and document every single walking distance, while timers add added convenience for various tasks and standard tests which have a time component.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

Referring now to the drawings in which like reference numbers represent corresponding parts throughout:

FIG. 1 illustrates one potential embodiment walker gait tracker according to the present invention.

FIGS. 2a-b illustrate additional potential embodiments for electronics within a walker gait tracker according to the present invention.

FIG. 3 illustrates a schematic of a digital logic components for implementing walker gait tracker according to the present invention.

FIG. 4 illustrates a flowchart for operating a walker gait tracker according to the present invention.

FIG. 5 illustrates a generalized schematic of a programmable processing system utilized as the various computing components described herein used to implement an embodiment of the present invention.

### DETAILED DESCRIPTION

This application relates in general a system and method that attempts to address the existing limitations in measuring a patient's gait while using a walker.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps, or components but do not preclude the presence or addition of one or more other features, steps, or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “50-250 micrometers should be interpreted to include not only the explicitly recited values of about 50 micrometers and 250 micrometers, but also include individual values and sub-ranges within the indicated range. Thus, included in this numerical range are individual values such as 60, 70, and 80 micrometers, and sub-ranges such as from 50-100 micrometers, from 100-200, and from 100-250 micrometers, etc.

As used herein, the term “about” means that dimensions, sizes, formulations, parameters, shapes and other quantities and characteristics are not and need not be exact, but may be approximated and/or larger or smaller, as desired, reflecting tolerances, conversion factors, rounding off, measurement error and the like and other factors known to those of skill. Further, unless otherwise stated, the term “about” shall expressly include “exactly,” consistent with the discussion above regarding ranges and numerical data.

The term “user” and “patient” refers to an entity, e.g. a human, that operates an automobile according to the present invention in order to park a car in a smart parking. For such a user, the terms “user” and “driver” may be used herein interchangeably.

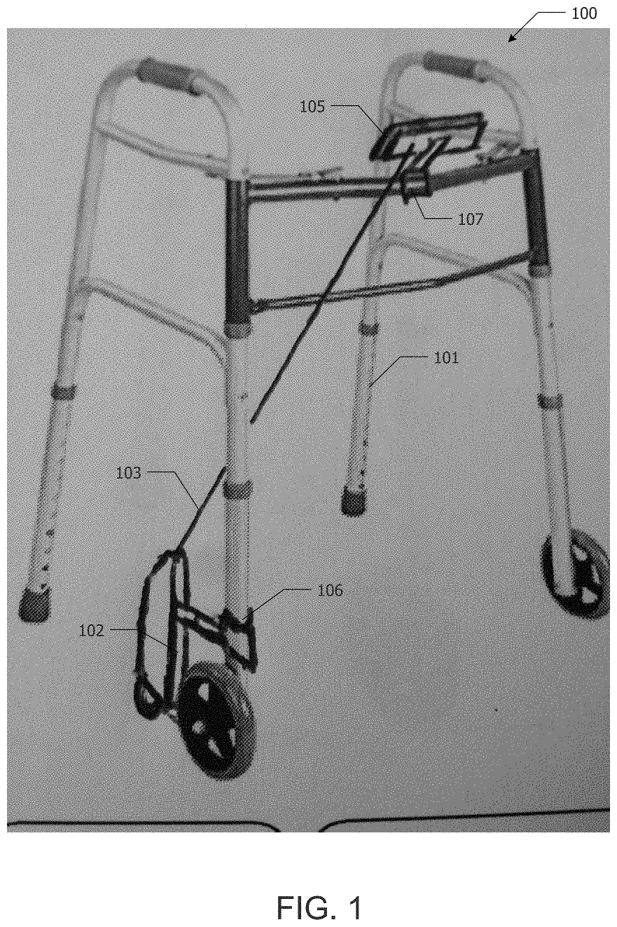

FIG. 1 illustrates one potential embodiment walker gait tracker according to the present invention. Gait Tracker 100 provides the answer to these inefficiencies and inaccuracies, which are very common in physical therapy situations. Gait Tracker 100 consists of 3 components: a walker mountable distance measuring wheel 102 having a clamp 106 for coupling to a wheel, a cord for transmission of distance data 103 to control and display console 105, and the control and display console 105 which is also walker mountable 107, device control and LED display console.

Gait Tracker 100 offer the therapist convenience and accuracy. This Gait tracker 100 also provides the patient with real-time information which can which can facilitate greater motivation and effort toward reaching specific goals. Patients are likely to make an extra effort to beat a specific time, or walk a specific distance with their walker, when they are able to see real-time distance and time information. Gait Tracker 100 is equipped with adjustable clamps for components 102 and 105, as well as multiple, adjustable pivot/swivel points 132, allowing it to accommodate different patients and be easily mounted and removed from various types of walkers. The control and display console 105 also offers a control switch for selection of Standard US measures, and for Metric measures.

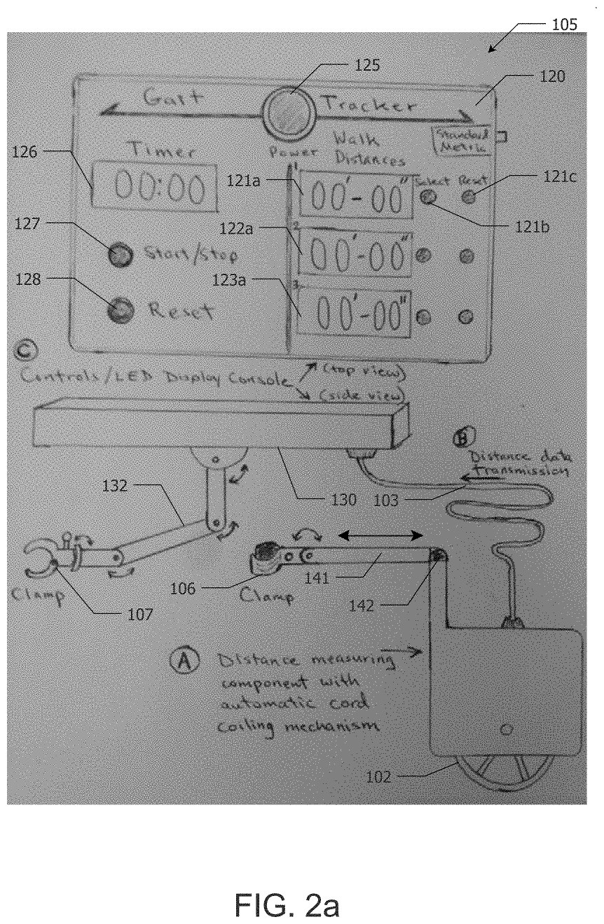

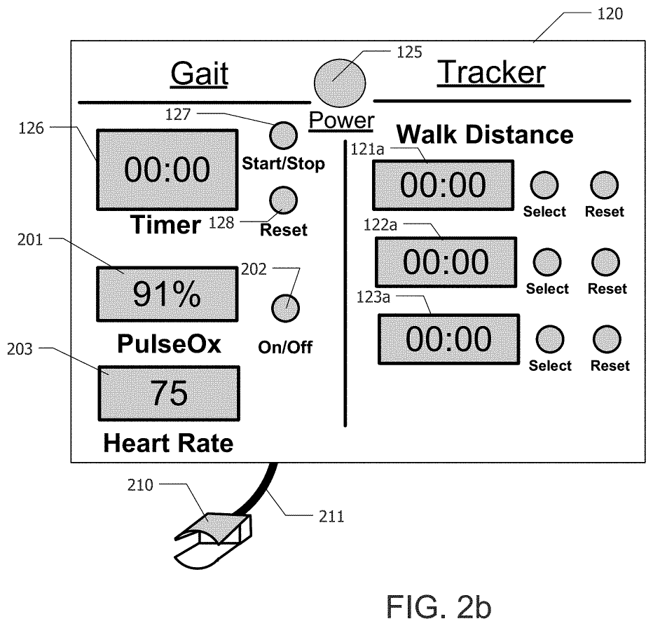

FIG. 2a illustrate another potential embodiment for electronics within a walker gait tracker according to the present invention. The control and display console 105 consists of a power button 125, multiple distance display boxes 121a, 122a, 123a for separate measurement and distance displays of multiple walks. Each display box 121a, 122a, 123a has its own Select button 121b for activation of distance display for the corresponding walk.

For example, therapist would press Select 1 121b at display box 1 121a for first walk, Select 2 122b for display box 2 122a for second walk, and Select 3 123b for display box 3 123a for the third walk, with distances of each, individual walk left on display, until the corresponding Reset button box 121c, 122c, 123c is pushed, returning that display boxes box 121a, 122a, 123a to zero (00:00) after the therapist has recorded the respective distances.

Gait Tracker 100 also features a simple timer 126, which can be used for multiple, frequently used, standard tests which involve a time component. The timer has a Start/Stop button 127 for starting and stopping the timer, as well as a Reset button 128 to reset timer 126 back to zero (00:00).

FIG. 2a also illustrates the various mounting mechanisms for attaching the measuring wheel 102 and its supports onto a walker 101. The measuring wheel 102 rotates as the walker is moved and it generates a pulse for each equal segment of the circumference of the measuring wheel 102. This component is mounted on a leg of the walker 101 at a height that allows the wheel to rest on the floor and freely rotate when moved. In one embodiment, the measuring wheel is attached to the walker using a clamp 106 that I coupled to a support arm 141. The clamp 104 may be coupled around a leg and the height may be controlled using a pivot joint at the clamp 104. In a first embodiment, the support arm 141 may be affixed to the measuring wheel assembly 102 using a fixed joint. In alternate embodiments the support arm 141 may pivot 142 up and down at the point where the arm attaches to the assembly. Additionally, the support arm 141 may also extend or telescope inward and outward to assist in orienting the measuring wheel 102 regardless of the shape of a walker's legs.

FIG. 2b illustrates another potential embodiment for electronics within a walker gait tracker according to the present invention. A pulse oximeter feature involving a short cord 212 with a sensor 211 at the end of it has been added to the console of FIG. 2a. The pulse-ox sensor 211 plugs into one of the side of the display console 105 using a cord 212 and displays a heart rate and a blood-oxygen saturation level of the patient. Such pulse oximeters are often used in rehab settings, especially for cardiovascular compromised patients which are on supplemental oxygen and need to maintain/exceed a specific, minimal blood-oxygen saturation level.

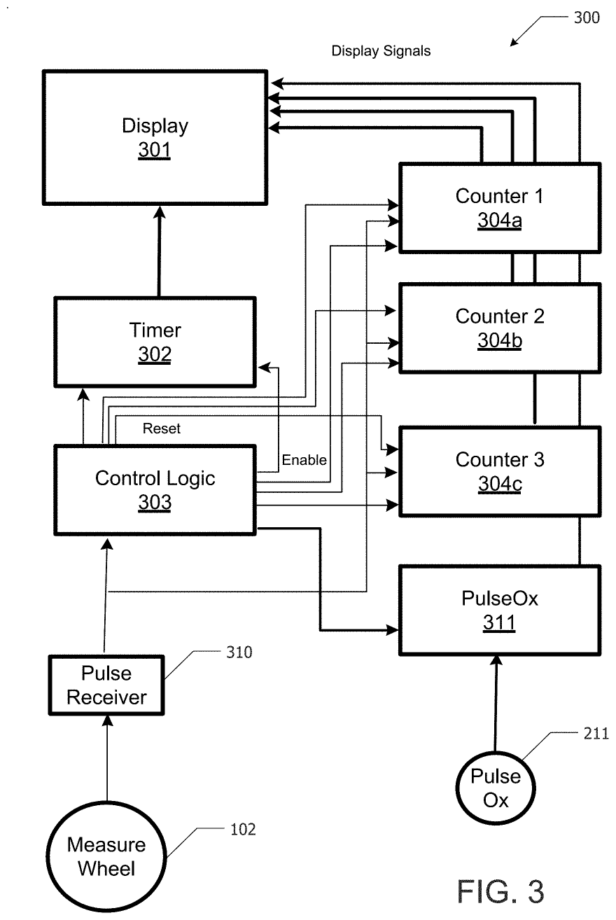

FIG. 3 illustrates a schematic of a digital logic components for implementing walker gait tracker according to the present invention. The control and display console 105 consists of a plurality of counters 304a-c, each of which receives an enable signal and a reset signal, control logic 303, a timer 302, a pulse receiver 310 coupled to the measuring wheel 102 by a data cable 103, a pulse-ox display 201, a heart rate display 203, and a pulse-ox sensor 211.

The control logic 303 receives signals from the console display of FIG. 2a above to generate the enable and reset signals that control the counters. When any of the counters are enabled, the counter 304a will increment itself upon receipt of each signal from the pulse receiver 310. This counting operation continues as long as the tracker is enabled. The control and display console 105 may calculate and display a measurement of the distance traveled on the display of the control and display console 105.

The timer 302 is enabled and reset using signals from control logic 303 and counts a fixed length of time that the timer 302 is enabled. Using this timer 302 and the counter 304a to measure distance traveled permits a therapist to calculate a rate of travel for a patient using the walker gait tracker 100.

The Pulse-Ox module 311 receives a signal from a sensor 211 that is typically attached to a patient's finger and the pulse-ox module generates a blood-oxygen saturation level and a heart rate value which are displayed on the pulse-ox display 201 and the heart rate display 203 respectively. Various embodiments may include the pulse-ox sensor 211 and pulse-ox module 311 where simpler models may not.

The use of the timers and pulse-ox module 311 permits the walker gait tracker to support additional uses. For example, There are a couple of common standardized tests which involve both distance and time components. One is called the “Timed Up and Go Test” (TUG). A patient is timed in performing the task of standing up from a chair, walking straight ahead 10 feet, pulling a U-turn at that 10 foot point, walking back to the chair and sitting back down. As a patient's time to perform this decreases (as a result of their PT), it implies greater strength, better balance and greater overall ability to get up and around safely. The vast majority of patients we do this test with, use a walker of some sort.

Another relatively common test is the “6 minute walk test”. With this one, the patient must walk as far as they can for 6 minutes. Increasing distance reached in 6 minutes, over time implies improving strength, mobility, balance and cardiovascular endurance. When starting physical therapy, these test or other time-distance based tests can establish starting baseline and then therapy goals can be set up toward reaching specific time and/or distance goals which would show improvements in strength and mobility. Another simple use of timer could be with the development of the standing tolerance of very low level patients. A patient too weak to stand up at all, could have a goal of eventually simply standing up at a walker for 15, 30, 60 . . . seconds before needing to sit down. Pulse-oximeter can be used on any patient which may need to have their heart rate and/or blood oxygen levels monitored. Cardiac rehab patients are supposed to follow specific protocols involving specific target heart rate ranges. The National Institute of Health has outlined a set of Cardiac Rehab Heart Rate Guidelines as follows:

BACKGROUND: One of the well-established methods used to determine endurance training intensity for patients in outpatient cardiac rehabilitation (CR) is to take a percentage (70%-85%) of the maximal or peak heart rate (HRmax) from a recent post-event symptom-limited graded exercise test (GXT).

Some patients have heart failure or lung issues like COPD, which can leave them susceptible to having their blood oxygen level fall below what is generally considered to the safe minimum of 90% to 92%. Some people will need to have this monitored, especially during activity like walking, so that we know if we need to get the patient on supplemental oxygen, or increase the rate of oxygen if they are already on it.

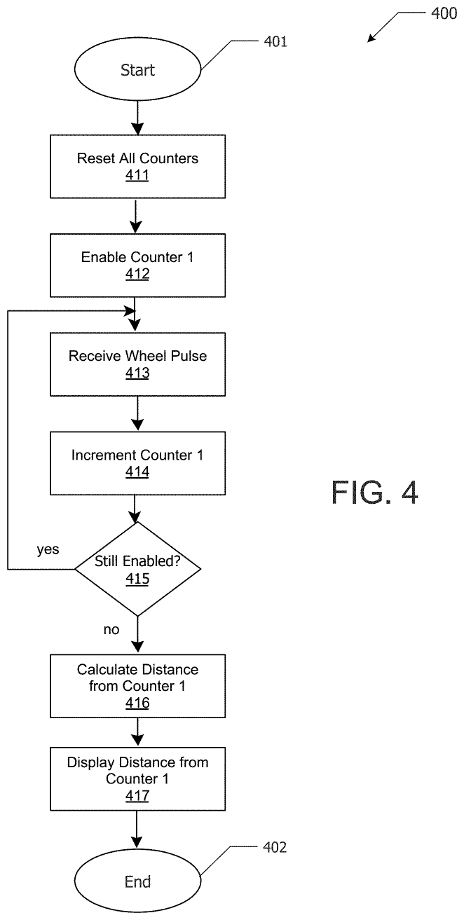

FIG. 4 illustrates a flowchart for operating a walker gait tracker according to the present invention. This embodiment of a method 400 describes the process steps used for walking a patient with a walker gait tracker 100 using one of the plurality of counters 304a. Of course, multiple legs of a longer walk made by the patient may be made using a series of different counters 304a-c one after the other.

The process begins 400 and a counter 304a is reset to zero in step 411. The counter 304a is enabled in step 412. When a wheel pulse is received by the pulse receiver 310, step 413 receives and detects this signal. The counter 304a is incremented in step 414 before test step 415 determines if the tracker 100 is still enabled to measure a distance. If test step 415 determines that the tracker 100 is still enabled, the process returns to step 413 to await the next wheel pulse. If test step 415 determines that the tracker 100 is no longer enabled, step 416 calculates the distance traveled using the counter's 304a value for the number of wheel pulses received and a circumference value for the measuring wheel 102. The calculated distance may be displayed by step 417 onto the control and display 105 console before the process ends 402.

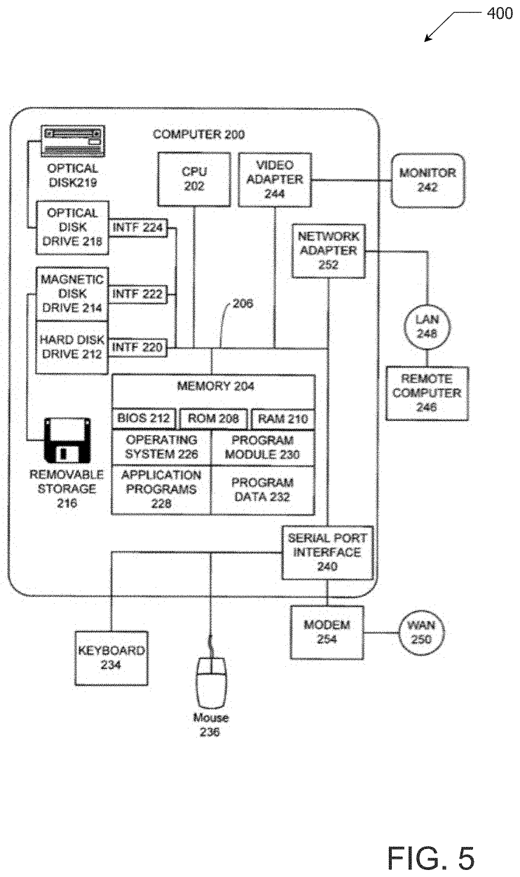

FIG. 5 illustrates a generalized schematic of a programmable processing system utilized as the various computing components described herein used to implement an embodiment of the present invention. The method disclosed herein and the functionality of the digital logic components of FIG. 3 may be implemented as a collection of software elements. These software elements may be executed using a single-chip microcontroller or a more general purpose computing device. For example, the software elements corresponding to digital logic components of FIG. 3 may be executed and displayed on a smartphone or small tablet computer. In all of these cases, these general purpose computing devices may be represented as a computing system 200 of FIG. 5.

The central processing unit (“CPU”) 202 is coupled to the system bus 204. The CPU 202 may be a general-purpose CPU or microprocessor, graphics processing unit (“GPU”), and/or microcontroller. The present embodiments are not restricted by the architecture of the CPU 202 so long as the CPU 202, whether directly or indirectly, supports the operations as described herein. The CPU 202 may execute the various logical instructions according to the present embodiments.

The computer system 200 also may include random access memory (RANI) 208, which may be synchronous RAM (SRAM), dynamic RAM (DRAM), synchronous dynamic RANI (SDRAM), or the like. The computer system 200 may utilize RAM 208 to store the various data structures used by a software application. The computer system 200 may also include read only memory (ROM) 206 which may be PROM, EPROM, EEPROM, optical storage, or the like. The ROM may store configuration information for booting the computer system 200. The RAM 208 and the ROM 206 hold user and system data, and both the RAM 208 and the ROM 206 may be randomly accessed.

The computer system 200 may also include an input/output (I/O) adapter 210, a communications adapter 214, a user interface adapter 216, and a display adapter 222. The I/O adapter 210 and/or the user interface adapter 216 may, in certain embodiments, enable a user to interact with the computer system 200. In a further embodiment, the display adapter 222 may display a graphical user interface (GUI) associated with a software or web-based application on a display device 224, such as a monitor or touch screen.

The I/O adapter 210 may couple one or more storage devices 212, such as one or more of a hard drive, a solid-state storage device, a flash drive, a compact disc (CD) drive, a floppy disk drive, and a tape drive, to the computer system 200. According to one embodiment, the data storage 212 may be a separate server coupled to the computer system 200 through a network connection to the I/O adapter 210. The communications adapter 214 may be adapted to couple the computer system 200 to the network 208, which may be one or more of a LAN, WAN, and/or the Internet. The communications adapter 214 may also be adapted to couple the computer system 200 to other networks such as a global positioning system (GPS) or a Bluetooth network. The user interface adapter 216 couples user input devices, such as a keyboard 220, a pointing device 218, and/or a touch screen (not shown) to the computer system 200. The keyboard 220 may be an on-screen keyboard displayed on a touch panel. Additional devices (not shown) such as a camera, microphone, video camera, accelerometer, compass, and or gyroscope may be coupled to the user interface adapter 216. The display adapter 222 may be driven by the CPU 202 to control the display on the display device 224. Any of the devices 202-222 may be physical and/or logical.

The applications of the present disclosure are not limited to the architecture of computer system 200. Rather the computer system 200 is provided as an example of one type of computing device that may be adapted to perform the functions of a parking management system, including servers, personal computers, and mobile devices as shown in FIG. 3. For example, any suitable processor-based device may be utilized including, without limitation, personal data assistants (PDAs), tablet computers, smartphones, computer game consoles, and multi-processor servers. Moreover, the systems and methods of the present disclosure may be implemented on application specific integrated circuits (ASIC), very large scale integrated (VLSI) circuits, or other circuitry. In fact, persons of ordinary skill in the art may utilize any number of suitable structures capable of executing logical operations according to the described embodiments. For example, the computer system 200 may be virtualized for access by multiple users and/or applications.

Additionally, the embodiments described herein are implemented as logical operations performed by a computer. The logical operations of these various embodiments of the present invention are implemented (1) as a sequence of computer implemented steps or program modules running on a computing system and/or (2) as interconnected machine modules or hardware logic within the computing system. The implementation is a matter of choice dependent on the performance requirements of the computing system implementing the invention. Accordingly, the logical operations making up the embodiments of the invention described herein can be variously referred to as operations, steps, or modules.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention. This written description provides an illustrative explanation and/or account of the present invention. It may be possible to deliver equivalent benefits using variations of the specific embodiments, without departing from the inventive concept. This description and these drawings, therefore, are to be regarded as illustrative and not restrictive.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in the testing measurements.

It will be further understood that various changes in the details, materials, and arrangements of the parts which have been described and illustrated in order to explain embodiments of this invention may be made by those skilled in the art without departing from embodiments of the invention encompassed by the following claims.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

What is claimed is:

1. A system for measuring a patient's gait while using a walker, the system comprises:
a console display unit having a clamping mechanism for attaching to the walker, the console display comprises:
a plurality of distance displays;
a plurality of counters each coupled to one of the plurality of distance displays;
a timer display;
a timer circuit having a start/stop input; and
control logic;
a measuring wheel assembly, having a measuring wheel with a known circumference and attachment clamp coupled to the assembly, coupled to a leg of the walker at a height that allows the measuring wheel to rest on the floor and rotate freely, the measuring wheel assembly generates an electronic pulse corresponding to a rotation of plurality of equal length segments on the circumference of the measuring wheel; and
one or more distance measurements based upon a number of electronic pluses received from the measuring wheel assembly and the known circumference of the measuring wheel;
wherein only one of the plurality of counters and corresponding distance display are active at any one time.

2. The system according to claim 1, wherein the system further comprises:
a pulse-ox sensor coupled to a pulse-ox and heart rate circuit within the console display; and
the console display further comprises the pulse-ox and heart rate circuit coupled to a pulse-ox display and a heart rate display.

3. The system according to claim 1, each of the counters and corresponding displays have a select button to enable the corresponding counter and display, and a reset button to set the counter to zero.

4. The system according to claim 3, wherein the plurality of counters and corresponding displays comprise 3 counter-display pairs.

5. The system according to claim 1, wherein the timer has a start/stop button and a reset button, and the timer measure passage of time in minutes and seconds.

6. A method for measuring a patient's gait while using a tracker coupled to a walker, the method comprising:
resetting all counters to zero;
enabling one counter;
receiving a plurality of electronic pulses from a measuring wheel assembly, the measuring wheel assembly having a measuring wheel with a known circumference and attachment clamp coupled to the assembly; and
displaying a distance traveled based upon a number of electronic pluses received from the measuring wheel assembly and the known circumference of the measuring wheel.

7. The method according to claim 6, wherein the method further comprises:
receiving a signal from a pulse-ox sensor;
determining a blood-oxygen saturation level and a heart rate value from the received signal;
displaying the blood-oxygen saturation level on a pulse-ox display; and
displaying the heart rate on a heart rate display.
